(19) United States
(12) Patent Application Publication
(10) Pub. No.: US 2018/0101653 A1
(43) Pub. Date: Apr. 12, 2018

(54) PET BLOOD TESTER, PET BLOOD TEST SYSTEM, AND PET BLOOD TESTING METHOD THEREOF

(71) Applicant: SAMSUNG ELECTRONICS CO., LTD., Suwon-si (KR)

(72) Inventors: Ki Ju LEE, Suwon-si (KR); Yu Ri SON, Gunpo-si (KR); Keum Yong OH, Yongin-si (KR)

(73) Assignee: SAMSUNG ELECTRONICS CO., LTD., Suwon-si (KR)

(21) Appl. No.: 15/664,306

(22) Filed: Jul. 31, 2017

(30) Foreign Application Priority Data
Oct. 11, 2016 (KR) 10-2016-0131423

(51) Int. Cl. G06F 19/00; A01K 11/00; A01K 29/00; G01N 33/49

(52) U.S. Cl. CPC G06F 19/345; A01K 11/006; G06F 19/322; G01N 33/49; A01K 29/00

(57) ABSTRACT

A pet blood tester includes a reader configured to read identification information of a pet from a tag; an analyzer configured to analyze blood of the pet to acquire information on the blood; a storage; a controller configured to receive stored body information of the pet corresponding to the identification information from at least one of an external device and the storage if the stored body information of the pet corresponding to the identification information is stored in at least one of the external device and the storage; and an inputter configured to receive input body information of the pet from a user if the body information of the pet corresponding to the identification information is not stored in at least one of the external device and the storage, wherein the controller is further configured to provide a blood test result and diagnosis information for the pet, based on the information on the blood, and the stored body information of the pet or the input body information on the blood.

### CROSS-REFERENCE TO RELATED APPLICATION

This application claims priority from Korean Patent Application No. 10-2016-0131423, filed on Oct. 11, 2016 in the Korean Intellectual Property Office, the disclosure of which is incorporated herein by reference.

### BACKGROUND

### 1. Field

The present disclosure relates to a pet blood tester and a control method thereof, and more particularly, to a technology that provides a user with a state of progress and result of a pet blood test based on pet body information received from an external device or the user and information on analyzed pet blood.

### 2. Description of the Related Art

In a veterinary clinic, a veterinarian performs various tests using clinical pathology test equipment to diagnose or collect information on a disease of a pet, such as a dog, cat or other animal. A blood test is used as one of the various tests.

A blood test is a method of determining a pet's current health condition by analyzing a small amount of blood collected from the pet. Because information on a pet can be easily acquired by a relatively simple method such as blood collection, a blood test is often used as a method of diagnosing a pet's disease.

However, because various types of pets exist, the type and characteristics of a pet should be accurately known to perform a blood test on the pet.

For example, when “cat” is input into a pet blood tester, but a pet from which blood is actually collected is a dog, an incorrect test result may be shown. Therefore, information on a pet should be accurately indicated to perform a blood test on the pet.

However, according to the related art, because a user has to directly input information on a pet before a blood test is performed, a problem arises in that information on a pet input by the user is different from information on an actually tested pet. In this case, an incorrect test result may be shown to the user, which leads to misdiagnosis of the pet and incorrect treatment of the pet.

Also, in the related art, when a veterinary clinic in which a pet is treated is changed, because body information of the pet that a previous clinic has cannot be used, there is an inconvenience that arises because a user has to re-input information on the pet, and because previous treatment results of a pet also cannot be used, treatment of the pet becomes inefficient.

### SUMMARY

Exemplary embodiments provide a device, system and method that can automatically receive information on a pet on which a blood test will be performed by using a wireless technology such as a tag and perform a blood test on the pet on the basis of the received information so that a blood test result and information on the pet are more easily provided to a user.

According to an aspect of an exemplary embodiment, there is provided a pet blood tester including: a reader configured to read identification information of a pet from a tag; an analyzer configured to analyze blood of the pet to acquire information on the blood; a storage; a controller configured to receive stored body information of the pet corresponding to the identification information from at least one of an external device and the storage if the stored body information of the pet corresponding to the identification information is stored in at least one of the external device and the storage; and an inputter configured to receive input body information of the pet from a user if the body information of the pet corresponding to the identification information is not stored in at least one of the external device and the storage, wherein the controller is further configured to provide a blood test result and diagnosis information for the pet, based on the information on the blood, and the stored body information of the pet or the input body information on the blood.

The controller may be further configured to receive the stored body information of the pet that is most recently stored or is selected according to a standard preset by the user, when the same stored body information of the pet is stored in the external device and the storage.

The controller may be further configured to control the external device to store in the external device the blood test result of the pet, and the input body information of the pet or the stored body information of the pet stored in the storage.

The body information may include at least one of a name, a species, a sex, a blood type, an age, a height, and a weight of the pet.

The blood test result may include at least one of a blood type test result, a clinical chemistry test result, and an electrolyte test result.

The identification information may include at least one of a barcode, a quick response code, text data, radio frequency identification information, and a recognition pattern.

The reader may include at least one of a barcode reader, a radio frequency identification reader, a near-field communication sensor, and an image sensor.

The storage may store the input body information received from the user or information received from the external device.

The pet blood tester may further include a display configured to display a state of progress of analysis or to display at least one of the blood test result and the diagnosis information.

The controller may be further configured to receive, from the external device, information on another pet present in a veterinary clinic within a predetermined area.

The controller may be further configured to determine whether a blood donation from the pet to the other pet or a blood transfusion to the pet from the other pet is possible based on the information on the other pet received from the external device.

The controller may be further configured to provide at least one of a blood type map, disease information, and clinic information of at least one pet on based on the information on the other pet received from the external device.

According to an aspect of another exemplary embodiment, there is provided a control method of a pet blood tester, the control method including: reading identification information of a pet from a tag; analyzing blood to acquire information on blood of the pet; receiving stored body information of the pet corresponding to the identification information if the stored body information of the pet corresponding to the identification information is stored in at least one of an external device and a storage of the pet blood tester; receiving input body information of the pet from a user if the stored body information is not stored in at least one of the external device and the storage of the pet blood test; and providing the user with a blood test result and diagnosis information for the pet based on the information on the blood, and the stored body information of the pet or the input body information of the pet from the user.

The control method may further include storing the input body information of the pet and the blood test result of the pet.

The body information may include at least one of a name, a species, a sex, a blood type, an age, a height, and a weight of the pet.

The control method may further include determining whether a blood donation from the pet to another pet or a blood transfusion to the pet from the other pet is possible based on blood type information of the pet and blood type information of the other pet that is stored in the external device.

The reading of the identification information may include reading the identification information with at least one of a barcode reader, a radio frequency identification reader, a near-field communication sensor, and an image sensor.

The control method may further include displaying a state of progress of the analyzing or displaying at least one of the blood test result and the diagnosis information.

The receiving the stored body information of the pet may include receiving, from the external device, information on a species, a sex, a blood type, a blood donation, and a blood transfusion of another pet present in a veterinary clinic within a predetermined area.

The control method may further include providing the user with information indicating whether a blood donation from the pet to the other pet or a blood transfusion to the pet from the other pet is possible based on the information on the other pet received from the external device.

### BRIEF DESCRIPTION OF THE DRAWINGS

The above and/or other aspects will become apparent and more readily appreciated from the following description of exemplary embodiments, taken in conjunction with the accompanying drawings of which:

FIG. 1 is a view illustrating an exterior of a pet blood tester according to an exemplary embodiment;

FIG. 2 is a block diagram illustrating a configuration of the pet blood tester according to an exemplary embodiment;

FIG. 3 is a view schematically illustrating an operation flow of the pet blood tester according to an exemplary embodiment;

FIG. 4 is a flowchart illustrating a control method of a pet blood tester according to an exemplary embodiment;

FIG. 5 is a view illustrating an example in which a reader reads identification information of a pet;

FIG. 6 is a view illustrating a state in which body information of a pet received from an external device by a user is displayed on a display according to an exemplary embodiment;

FIG. 7A is a view illustrating a state in which the user indicates that it is necessary to input body information of a new pet through an inputter according to an exemplary embodiment;

FIG. 7B is a view illustrating a state in which the user inputs body information of a new pet through an inputter according to an exemplary embodiment;

FIG. 8 is a view illustrating a state in which a start process of a blood test is displayed on the display according to an exemplary embodiment;

FIG. 9 is a view illustrating a state in which a state of progress in a blood test is displayed on the display according to an exemplary embodiment;

FIG. 10 is a view illustrating a state in which a blood test result is displayed on the display according to an exemplary embodiment;

FIG. 11 is a view illustrating a state in which a list of blood tests are displayed on the display according to an exemplary embodiment;

FIG. 12 is a block diagram illustrating a configuration of a pet blood test system according to another exemplary embodiment;

FIG. 13 is a view illustrating a state in which a blood type map of pets is displayed on the display according to an exemplary embodiment.

FIG. 14 is a view illustrating a state in which a disease map of pets is displayed on the display according to an exemplary embodiment; and

FIG. 15 is a flowchart illustrating a control method of a pet blood test system according to still another exemplary embodiment.

### DETAILED DESCRIPTION

Embodiments disclosed herein and configurations illustrated in the drawings are examples, and various modified embodiments that may substitute the embodiments herein and the drawings may be made.

Terms used herein are for describing the exemplary embodiments and are not intended to limit and/or restrict the present disclosure. A singular expression includes a plural expression unless the context clearly indicates otherwise.

In the present specification, terms such as “include,” “equipped with” or “have” are for designating that features, number, steps, operations, elements, parts, or combinations thereof exist and do not preclude the existence of or the possibility of adding one or more other features, numbers, steps, operations, elements, parts, or combinations thereof in advance.

Terms including ordinals such as “first” and “second” may be used to describe various elements, but the elements are not limited by the terms. The terms are only used for the purpose of distinguishing one element from another element. For example, a first element may be referred to as a second element while not departing from the scope of the present disclosure, and likewise, a second element may also be referred to as a first element. The term “and/or: includes a combination of a plurality of related described items or any one item among the plurality of related described items.

Exemplary embodiments will be described in detail below with reference to the accompanying drawings so that one of ordinary skill in the art to which the present disclosure pertains is able to easily embody the present disclosure. Also, to clearly describe the present disclosure, parts unrelated to the description will be omitted from the drawings.

In the present specification, “user” is a medical expert and may be a doctor including a veterinarian, a nurse, a medical image specialist, etc. but is not limited thereto.

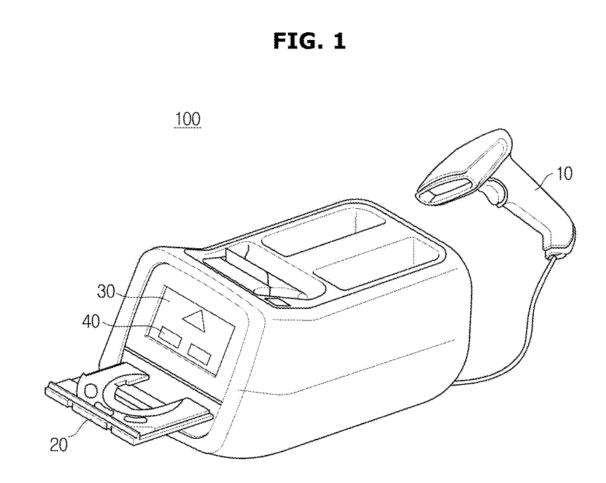

A pet blood tester 100 may be manufactured in a portable size as illustrated in FIG. 1, may be loaded in a transport vehicle such as an ambulance or a helicopter ambulance capable of moving a critically ill pet, and is not limited to a specific size.

FIG. 1 is a view illustrating an exterior of a pet blood tester according to an exemplary embodiment.

Referring to FIG. 1, the pet blood tester 100 according to an exemplary embodiment may include a reader 10, a loader 20, and a display 30.

Upon receiving a test medium that contains blood collected from a pet on a loading surface of the loader 20, the pet blood tester 100 may analyze the blood included in the test medium and provide the analyzed result as a blood test result to a user through the display 30.

The reader 10 may serve to scan identification information of a pet to be tested.

Identification information of a pet refers to a unique identifier (ID) from which body information of the pet can be recognized and refers to a unique number analogous to a social security number of a person.

In the case of a pet, such an ID is generally stored through a tag such as a label. Consequently, body information of a pet may be stored by an electronic tag capable of transmitting and receiving information by a quick response (QR) code, a barcode, a microchip, radio frequency identification (RFID), and short-range communication such as near field communication (NFC).

Therefore, the reader 10 may include a barcode reader capable of scanning a barcode and include at least one of a QR code reader, an RFID reader, an NFC reader, and an image sensor. Alternatively, the reader could include all of the preceding readers/sensors.

The loader 20 may receive a test medium. Here, the test medium may be a medium that contains blood which is a test object.

Specifically, the loader 20 may include a medium inserting device configured to receive the test medium, and the shape of the loader 20 may vary depending on the shape of a test medium.

In FIG. 1, a case in which the test medium has the shape of a disc is illustrated as an example. In this case, the loader 20 may include a disc tray configured to receive a disc of a particular shape as the medium inserting device.

The loader 20 may operate the disc tray so that the disc tray is drawn out and allow a disc to be loaded on the disc tray.

In another example, unlike that illustrated in FIG. 1, a test medium may have the shape of a cartridge. In this case, the loader 20 may have a shape capable of receiving a cartridge.

The display 30 may display various information such as a state of progress in a blood test and a blood test result to the outside. Specifically, the display 30 may include a display panel, and a predetermined screen may be displayed on the display panel.

Also, the display 30 may display a user interface screen required for a blood test, a screen that notifies of a state of progress in the blood test, a screen that notifies of a test result, or the like.

The display 30 may be formed with a single display panel or a touchscreen in which a display panel and a touch pad are combined.

A user interface 40 configured to receive a command from a user or display specific information may be generated on the display 30.

The user interface 40 may generate or output a user interface screen to receive a command or body information of a pet from the user to perform a specific test on the pet. Consequently, the user may input a specific command or information through the user interface screen.

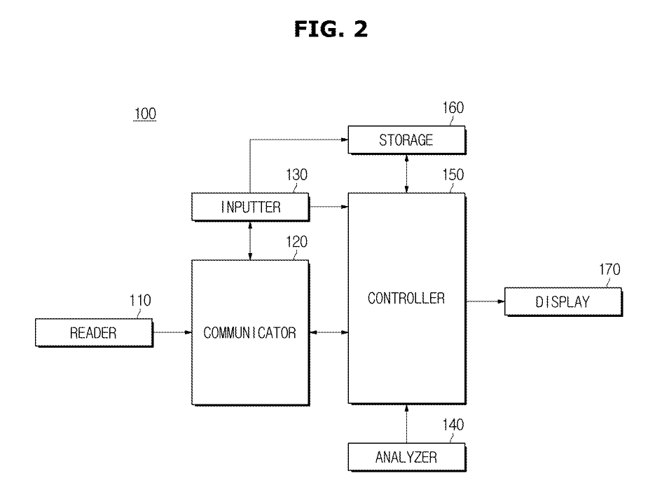

Additionally, the pet blood tester 100 may include an inputter 130 (see FIG. 2). The inputter may be implemented via a mouse, a keyboard, a joystick, or another type of input device that includes hard keys capable of inputting specific data.

Also, the user interface 40 may be formed with a touch pad. In this case, the user may input a specific command through the user interface screen, and the touch pad may sense the user's input and recognize the specific command input by the user.

Consequently, when the user touches a specific point on the user interface screen, the user interface 40 may sense the touched point and transmit sensed information to a controller 150. The controller 150 may be implemented by a processor (e.g., one or more programmed microprocessors and associated circuitry) and/or dedicated hardware, and may be programmed using software (e.g., microcode) to perform various functions discussed herein and may optionally be driven by firmware and/or software.

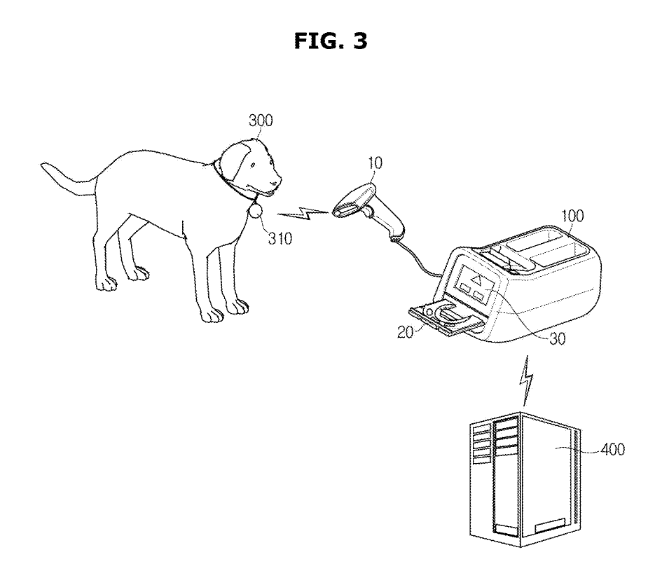

Then, the controller 150 may recognize the user's request or command corresponding to a menu displayed on the sensed point and perform the recognized request or command. The controller 150 will be described in detail below with reference to FIG. 3.

FIG. 2 is a block diagram illustrating a configuration of the pet blood tester 100 according to an exemplary embodiment.

Referring to FIG. 2, the pet blood tester 100 according to the present disclosure may include a reader 110 configured to read identification information of a pet, a communicator 120 configured to receive body information of the pet corresponding to the identification information of the pet from an external device, an inputter 130 configured to receive body information of the pet from a user when the body information of the pet is not present in the external device, an analyzer 140 configured to analyze blood collected from the pet and acquire information on the blood, the controller 150 configured to provide the user with a result of a desired type of blood test on the basis of the body information of the pet received by the communicator 120 and information on the analyzed blood, a storage 160 configured to store a blood analysis result and a blood test result, and a display 170 configured to display a state of progress in the blood test and a test result in the controller 150 to the outside.

The reader 110 may serve to acquire identification of a pet to be blood-tested.

Identification information of a pet refers to a unique ID from which body information of the pet can be recognized and refers to a unique number analogous to a social security number of a person.

For example, the identification information may include at least one of a name of a pet, a unique identification number of the pet, a blood identification number for identifying blood, the animal species, the medical history of the pet, age of the pet, gender of the pet, weight of the pet, vital statistics of the pet, contact information of the pet owner, etc.

The identification information of the pet may be stored in a code form or a tag form such as a label to be quickly scanned.

Consequently, the identification information may have a barcode form, a QR code form, an image data form, or text data form. Also, the identification information may be formed as radio signals that can be transmitted through short-range communication such as RFID or NFC.

The reader 110 may read unique identification information of a pet through the reader 10, and when identification information is read, may serve to transmit the read identification information to the communicator 120.

Consequently, the reader 110 may include the reader 10 that can play the above role, and because features of the reader 10 are the same as those described with reference to FIG. 1, the description thereof will be omitted.

After receiving the identification information of the pet transmitted from the reader 110, via, the communicator 120, the controller 150 performs a search to check whether the received identification information of the pet is stored in the external device.

When the identification information of the pet is present in the external device, the communicator 120 may serve to receive body information of the pet corresponding to the identification information of the pet and receive results of various tests which were previously performed on the pet. Also, the communicator 120 may serve to send the received information to the controller 150.

Consequently, the communicator 120 may be a transceiver or communication interface that includes one or more elements that enable communication, and for example, include at least one of a short range communication module, a wired communication module, and a wireless communication module. Each module may be implemented via application-specific integrated circuit (ASIC), software, or a combination of an ASIC and software.

The short range communication module may include various short range communication modules such as a Bluetooth module, an infrared communication module, an RFID communication module, a wireless local access network (WLAN) communication module, an NFC communication module, and a ZigBee communication module that are capable of transmitting and receiving a signal using a wireless communication network from a short range.

The wired communication module may not only include various wired communication modules such as a local area network (LAN) module, a wide area network (WAN) module, or a value added network (VAN) module, but also include various cable communication modules such as a universal serial bus (USB), a high definition multimedia interface (HDMI), a digital visual interface (DVI), a recommended standard 232 (RS-232), power line communication, or plain old telephone service (POTS).

The wireless communication module may not only include a wireless fidelity (Wi-Fi) module and a wireless broadband module but also include wireless communication modules that support various wireless communication methods such as global system for mobile communication (GSM), code division multiple access (CDMA), wideband CDMA (WCDMA), universal mobile telecommunications system (UMTS), time division multiple access (TDMA), and long term evolution (LTE).

The communicator 120 can receive body information of the pet from the external device. The communicator 120 may also receive body information of the pet stored in the storage 160 of the pet blood tester 100. When the information on the pet is not stored in the external device or connection with the external device is not smooth, because information on the pet can be directly received from the storage 160, the pet blood tester 100 can be more effectively used.

When the identification information of the pet is present in both the external device and the storage 160, to prevent collision between two pieces of information, the communicator 120 may select any one of the information stored in the external device and the information stored in the storage 160 and receive the information.

For example, the communicator 120 may receive information on the basis of most recently stored information, and when the user has preset a standard, e.g., has set the external device as a priority, the communicator 120 may receive information by placing priority on information from the external device even when the two pieces of information may collide with each other. When the user has set information stored in the storage 160 as priority, the communicator 120 may receive information by placing priority on the information stored in the storage 160.

When the body information of the pet corresponding to the identification information of the pet read by the reader 110 is not present in the external device, and the body information of the pet cannot be received by the communicator 120, the inputter 130 may serve to directly receive body information of the pet from the user. In other words, the user may manually input the pertinent information into the pet blood tester 100. Also, the inputter 130 may serve to receive a type of a blood test desired by the user.

When the user has previously stored the body information of the pet in the external device, receiving body information via the communicator 120 is easier than the user directly inputting body information of the pet.

However, when the body information of the pet is stored in another external device and the body information cannot be received or when a pet is blood-tested for the first time, because body information of the pet has to be newly input, the user may input the body information of the pet through the inputter 130 in this case.

Consequently, to be able to receive information from the user, the inputter 130 may include a mouse, a keyboard, a joystick, or another input device that includes hard keys capable of inputting specific data.

Also, the inputter 130 may be implemented as a touch pad or touch screen display. In this case, the user may input information or a command through the user interface screen, and the touch pad may sense the user's input, recognize the predetermined command input by the user, and then transmit the recognized information to the communicator 120.

When the inputter 130 is implemented as a touch pad, the display 170 that will be described below may simultaneously perform the role of the inputter 130.

The analyzer 140 may operate to analyze blood collected from a pet and obtain information on the blood.

Because blood includes numerous components, the analyzer 140 analyzes the collected blood to analyze which components are included in the blood and the amounts of each component. For example, components such as red blood cells, white blood cells, plasmas, and platelets and the amounts thereof may be analyzed.

The controller 150 may operate to perform a blood test on the basis of the body information of the pet received by the communicator 120 or the inputter 130 and the information on the blood of the pet acquired by the analyzer 140.

In a case of a blood analyzer for humans, a standard for performing a blood test is clear because the blood test performed on humans is usually standardized. However, in the case of pets, because various types of pets have different biochemistries and physiologies, a blood test on pets cannot be immediately performed only on the basis of the same blood components.

That is, a blood test should begin by setting “dog” as a blood test standard when a pet from which blood is collected is a dog, and a blood test should begin by setting “cat” as a blood test standard when a pet from which blood is collected is a cat. As a result, the testing can be suitable for the particular type of pet being tested or diagnosed.

Consequently, instead of immediately performing a blood test on the basis of the information on blood acquired by the analyzer 140, the controller 150 may perform a type of a blood test input by the user on the basis of the body information of the pet received by the communicator 120 or the inputter 130 and the information on blood acquired by the analyzer 140 and provide a result thereof to the user.

Also, the controller 150 may provide diagnosis information required for the pet on the basis of the blood test result of the pet. In this case, because the user can be aware of treatment, vaccination, and the like required for the pet in advance, there is an advantage in that treatment can be more efficiently performed on the pet.

The storage 160 may operate to store the body information of the pet received from the inputter 130 and the state of progress in blood analysis or test result obtained by the controller 150.

Although such pieces of information can be stored in the external device by the communicator 120, essential information of the pet is stored in the storage 160 so that the body information of the pet can be more easily and quickly acquired than when the body information of the pet is received from the external device.

Consequently, the storage 160 may include at least one type of storage medium among a flash memory type, a hard disk type, a multimedia card micro type, a card type memory (secure digital (SD), extreme digital (XD) memories, etc.) a random access memory (RAM), a static RAM (SRAM), a read-only memory (ROM), an electrically erasable programmable ROM (EEPROM), a PROM, a magnetic memory, a magnetic disk, and an optical disk.

The display 170 may operate to display a blood test result generated by the controller 150 to the outside so that the user can view the blood test result.

Consequently, the display 170 may include a display panel, and may be formed with a single digital panel or a touchscreen in which a display panel and a touch pad are combined.

The display 170 may display the user interface screen required for a blood test, a screen that notifies of a state of progress in the blood test, a screen that notifies of a test result, or the like, and the user interface 40 configured to receive a command from the user or display specific information may be generated.

Also, when the display 170 is configured in the form of a touchscreen, in addition to the display 170 displaying specific information to the outside, the user may input a specific command or information through the user interface screen.

The inputter 130 may include a mouse, a keyboard, a joystick, or an input device that includes hard keys capable of inputting specific data.

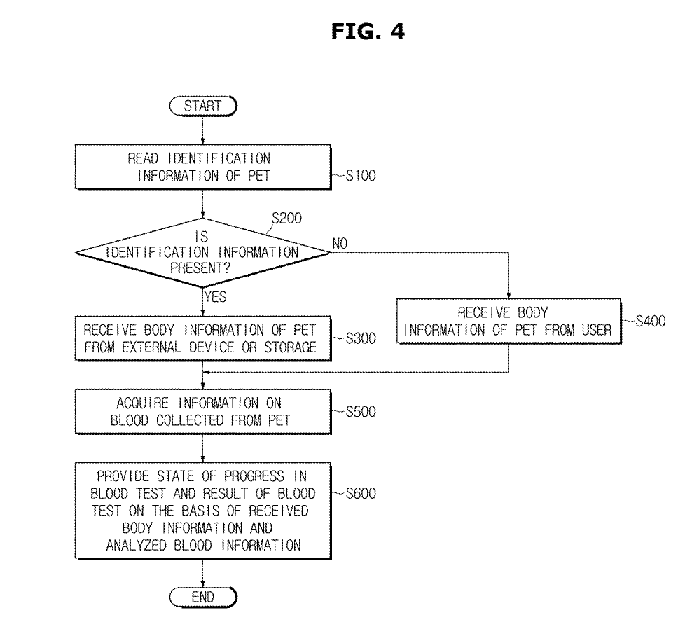

The pet blood tester 100 according to the present disclosure has been described above with reference to FIG. 2. An operation flow will be described below with reference to FIGS. 3 and 4.

FIG. 3 is a view schematically illustrating an operation flow of the pet blood tester 100, and FIG. 4 is a flowchart illustrating the operation flow of the pet blood tester 100.

Referring to FIG. 3, the pet blood tester 100 may read identification information of a pet 300 to be blood-tested by using the reader 10 and a tag 310.

The identification information of the pet refers to a unique number of the pet described with reference to FIG. 2, and the tag 310 may have a barcode form, a QR code form, an image data form, or a text data form.

When the identification information of the pet that is stored in the tag 310 is read by the reader 10, the communicator 120 may receive body information of the pet corresponding to the identification information of the pet from an external device 400 or the storage 160.

The external device 400 that has received the identification information of the pet may search for the body information of the pet corresponding to the identification information, and then transmit the body information of the pet back to the communicator 120. The external device 400 may be a cloud computing server that is located in an area remote from the pet blood tester 100.

When the body information of the pet is received by the communicator 120, the analyzer 140 may analyze blood collected from the pet which is loaded on the loader 20 and acquire information on the blood.

The controller 150 may perform a type of test input by the user on the basis of the information on the blood of the pet acquired by the analyzer 140 and the body information of the pet received by the communicator 120.

Also, when the test is finished, the controller 150 may transmit the test result to the display 170, and the display 170 may control the user interface according to the test result and provide the test result to the user.

The schematic operation flow has been described with reference to FIG. 3. Hereinafter, a control flow of the pet blood tester 100 will be described in detail with reference to the flowchart illustrated in FIG. 4.

When blood collected from a pet is prepared for testing, identification information of the pet to be blood-tested is read using a tag of the pet, and then whether the identification information of the pet is present is determined (S100, S200).

The identification information of the pet is read in order to obtain body information of the pet. When the identification information of the pet is obtained through the tag, the user can more easily acquire the body information of the pet.

When the identification information of the pet is read through the tag, the body information of the pet corresponding to the identification information of the pet is received from an external device (S300).

When the body information of the pet is received from the external device, because the body information of the pet can be more easily acquired, an inconvenience of having to input the body information manually every time a blood test is performed can be alleviated. Also, because results of various blood tests which were previously performed on the pet in the past can be acquired through a server, a current health condition of the pet can be more easily determined.

Also, even when a blood test is performed in another veterinary clinic, because body information of the pet can be acquired by an external device that is connected to the pet blood tester 100 by communication, a blood test can be more easily performed.

Because an object of a human blood tester is limited to humans and thus has a single standard, body information of a human does not have to be input in detail before a blood test is performed. However, in the case of pets, because various types of pets have different biochemistries and physiologies, body information of a pet has to be input to clarify the standard thereof before a blood test is performed.

The body information of the pet may include the pet's species, name, sex, blood type, age, height, weight, etc. but is not limited thereto, and when other information required for a blood test of the pet is present, the information thereon may also be included in the body information of the pet.

Also, a conventionally performed blood test may include a blood type test result, a clinical chemistry test result, an electrolyte test result, etc. but is not limited thereto, and other tests related to blood of the pet may also be included in the conventionally performed blood test.

When the identification information of the pet does not exist (S200—NO), the body information of the pet to be blood-tested may be received from the user (S400).

The received body information may be stored in the storage 160 or stored in the external device to be usefully used when attempting to perform a blood test again in the future.

When the body information of the pet is received, blood collected from the pet which is loaded on the loader 20 is analyzed to acquire information on the blood (S500).

In the case of pets, because various types of pets exist, a blood test should be performed after body information of a pet is input. This is because, otherwise, a different result may be produced on the basis of the same blood components.

For example, although a level of white blood cells of a dog may be determined to be abnormally low as a result of blood analysis of the dog, a level of white blood cells may not be determined to be abnormally low when blood is analyzed on the basis of a cat.

Consequently, because types and amounts of components that should be included in blood are different for each pet, to perform an accurate blood test, information on collected blood should be analyzed, and then received body information of the pet should be input to perform a blood test on the pet.

When information on the collected blood is acquired, a blood test is performed on the basis of the received body information and the analyzed blood information, and the state of progress in the blood test and the test result are provided to the user (S600).

The operation flow of the pet blood tester 100 according to the present disclosure have been described above in detail. Hereinafter, various exemplary embodiments of the pet blood tester will be described with reference to the drawings.

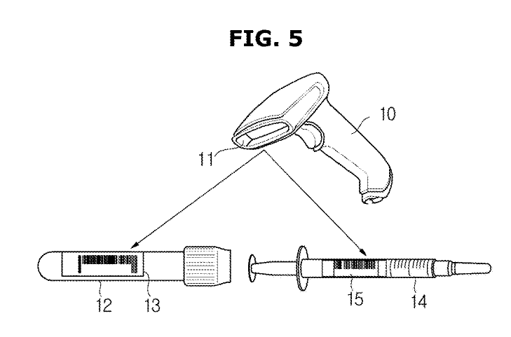

FIG. 5 is a view illustrating an example in which the reader 10 reads identification information of a pet

Because the reader 10 illustrated in FIG. 5 is the same as the reader 10 in FIG. 1, overlapping description with the description of FIG. 1 will be omitted.

When blood is collected from a pet, the collected blood may be dipped in a blood container 12. Although identification information of the pet may be attached to the pet in the form of a tag as illustrated in FIG. 3, identification information 13 may be attached to the blood container 12 as illustrated in FIG. 5. Also, identification information 15 may be attached to a syringe 14 used when blood is collected from the pet.

The reader 10 may include a scanner 11. The scanner 11 may scan barcodes (for example, 12, 13), and scanned information may be transmitted to the communicator 120.

When scanning the identification information of the pet is completed in the reader 10, the communicator 120 may receive body information of the pet corresponding to the identification information from an external device, and the analyzer 140 may begin blood analysis to acquire information on the blood.

Also, when scanning is finished, the controller 150 may control the loader 20 to be open so that the analyzer 140 can automatically perform blood analysis. Specifically, the controller 150 may operate the disc tray of the loader 20 to be drawn out and allow a blood test disc, which is a test medium, to be loaded on the disc tray.

Also, when the test medium is loaded on the disc tray, the controller 150 may control the loader 20 to be closed. When the loader 20 is completely closed, the controller 150 may control the analyzer 140 to begin analysis of the test object.

Also, the loader 20 may further include a sensor configured to sense insertion of a test medium. Specifically, the sensor may include a detection sensor configured to detect insertion of a test medium.

Here, the detection sensor is a sensor capable of recognizing that the test medium is loaded on the disc tray and may include a touch sensor, an infrared sensor, a pressure sensor, an ultrasonic sensor, etc. When the sensor includes an infrared sensor, a pressure sensor, an ultrasonic sensor, and the like, the sensor can recognize that the test medium is loaded on the disc tray.

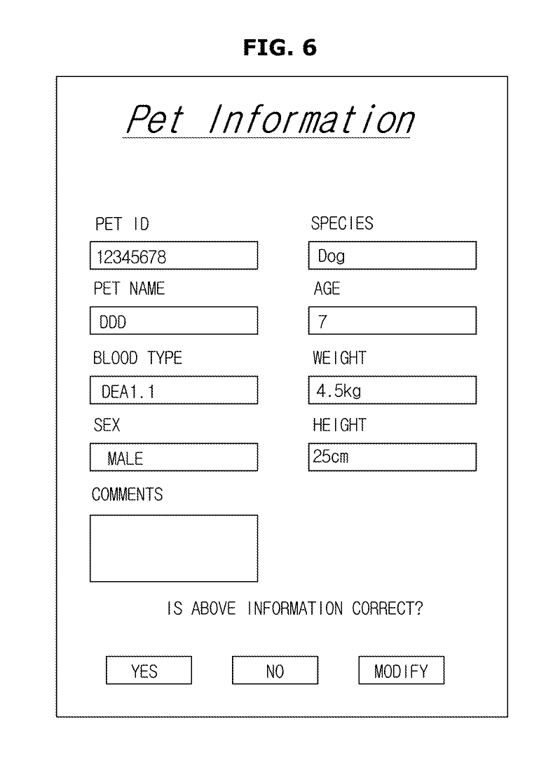

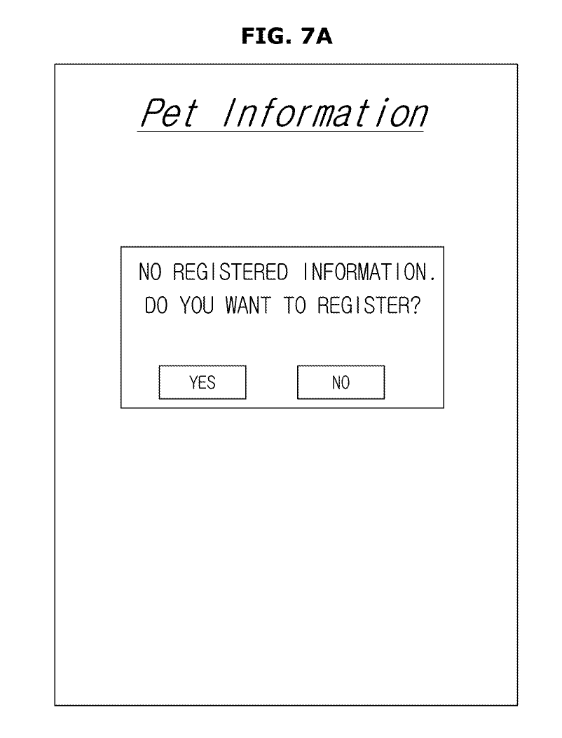

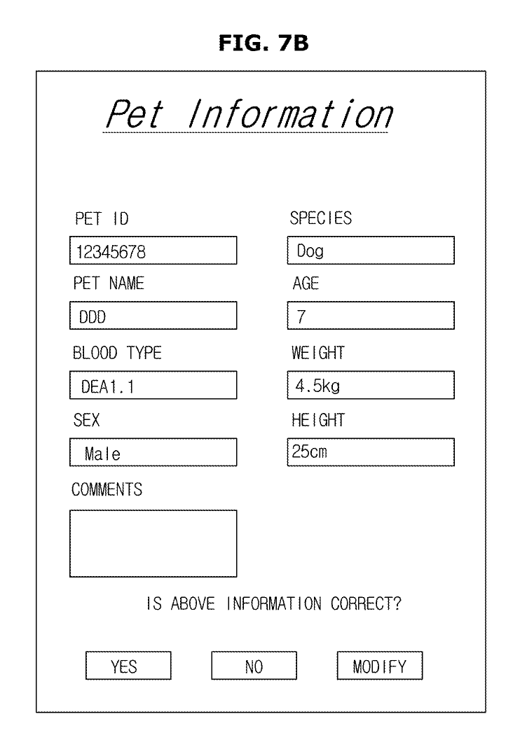

FIG. 6 is a view illustrating a state in which body information of a pet received from an external device by the communicator 120 is displayed on the display 170, and FIGS. 7A and 7B are view illustrating a state in which the user inputs body information of a pet through the inputter 130 through the display 170.

As shown in FIG. 6, the body information may include a pet ID number, a pet species, a pet name, a pet age, a blood type, a pet weight, gender, height, and stored comments. However, the body information is not limited to what is shown in FIG. 6. The body information could include other types of information, and certain data fields in FIG. 6 could be omitted.

When body information of a pet is received from an external device by the communicator 120, there is an advantage in that a blood test can be immediately performed without inputting the body information on the pet.

However, when a part in which body information of the pet does not match exists, the user may modify the body information of the pet through the inputter 130, and the modified information may be stored in an external device by the communicator 120 or stored in the storage 160 inside the pet blood tester 100.

When information on the pet does not exist in the external device, the user may input body information of the pet through the inputter 130 as illustrated in FIGS. 7A and 7B.

In FIG. 7A, the user indicates that body information should be associated with the pet. In FIG. 7B, the user enters the appropriate body information of the pet.

When the inputter 130 or the display 170 are formed of a touch pad, the user may use the touch pad to input body information of the pet, and the input information may be stored in an external device or stored in the storage 160 inside the pet blood tester 100.

When body information of the pet and the test result are stored in the storage 160, because information thereon can be easily received when a blood test is performed in the future, an inconvenience of having to input the body information again may be reduced.

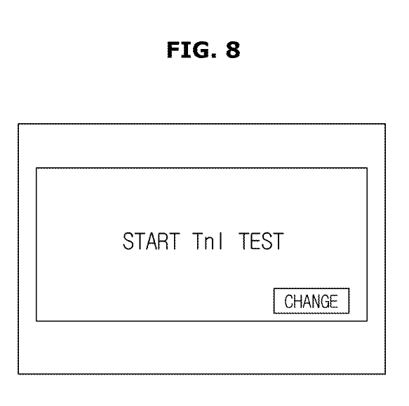

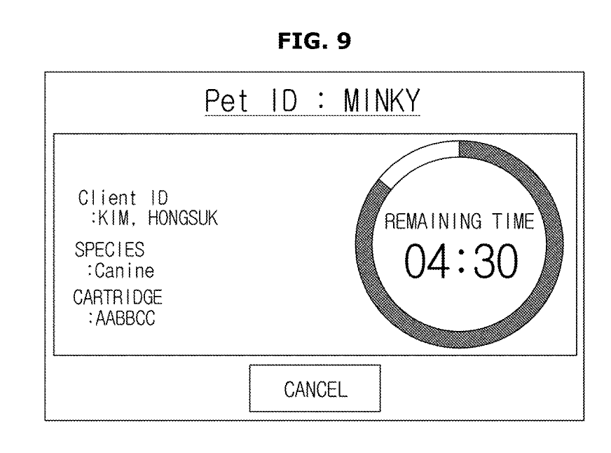

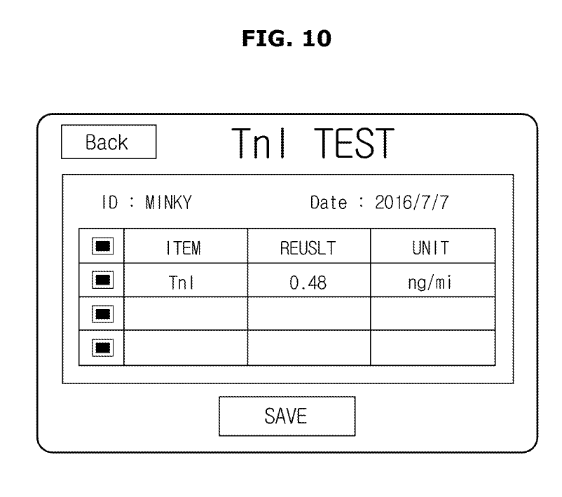

FIGS. 8 to 11 are views illustrating a state in which the state of progress in a blood test and a result of the blood test are displayed on the display 170. FIG. 8 is an exemplary embodiment of blood tests and illustrates a state in which a Troponin I (TnI) test begins, FIG. 9 illustrates a state in which the TnI test is in progress, and FIG. 10 is a view illustrating a test result after the TnI test is ended.

The TnI test is a cardiac marker test that is used to diagnose acute myocardial infarction (AMI). When a disease related to MI has occurred, the TnI test is required as a cardiac marker test. TnI is an index for myocardial injury and appears in blood when a myocardial tissue is damaged.

Although the TnI test is given as an example of a blood test in FIGS. 8 to 10, the blood test is not limited thereto and may include various types of blood tests including a blood type test, a clinical chemistry test, and an electrolyte test.

Referring to FIG. 8, the controller 150 may output a user interface screen that notifies of a start of a specific test on the display 170. Also, the user may use a test item changing menu included in the user interface screen to change a blood test item.

Also, the controller 150 may control a plurality of tests to be sequentially performed by initial setting. In succession, the controller 150 may control a user interface screen that notifies of the state of progress in a predetermined test to be output on the display 170.

Referring to FIG. 9, the display 170 may display a user interface screen that notifies of the state of progress in the TnI test. The user interface screen may include a menu screen that notifies a user of a test duration time, which is the state of progress in the TnI test, and a remaining test time until completion of the test.

Also, the user interface screen may further include a menu that allows progress of the pet blood test to be stopped or cancelled, e.g., a test cancel or test stop menu.

Consequently, even when the controller 150 automatically performs a test in response to completion of scanning, the execution of the corresponding test may be stopped or cancelled by reflecting a user's request.

Referring to FIG. 10, the display 170 may display a user interface screen that includes at least one blood test result, such as a TnI test, according to control by the controller 150.

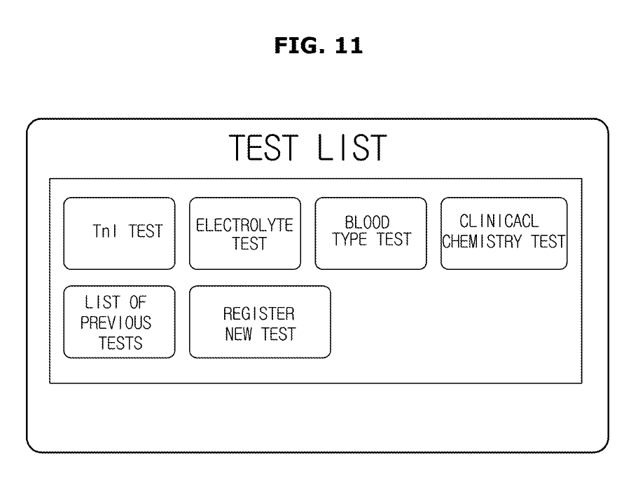

FIG. 11 is a view illustrating a screen on which a list of blood tests is displayed on the display 170 according to an exemplary embodiment.

Referring to FIG. 11, the display 170 may display test items that may be performed by the pet blood tester 100 or a list of blood test items which were previously performed. For example, the display 170 may display a TnI test, an electrolyte test, a blood type test, a clinical chemistry test, or all of the preceding tests. The display 170 may also display a list of previously performed tests. The display 170 may also receive input to register a new test, so that the pet blood tester 100 can perform the new test.

The user may load a previous test list using the inputter 130 or the display 170. When a new test is required, the user may order a new test and start the new test and store the test result in the storage 160 or an external device.

In the pet blood tester 100 described above with reference to the drawings, because body information of a pet can be automatically received from an external device, there is an advantage in that an inconvenience of having to input body information every time a blood test is performed can be alleviated.

Also, even when a blood test is performed in another veterinary clinic, because body information of the pet can be acquired by an external device that is connected by communication, a blood test can be more easily performed.

In this case, because body information of the pet can be automatically received and a blood test corresponding thereto can be performed instead of the body information being manually input by the user, the user's mistake can be prevented and a more accurate test result can be provided.

The configuration and features of the pet blood tester 100 according to an exemplary embodiment has been described above. Hereinafter, a pet blood test system 200 according to another exemplary embodiment will be described.

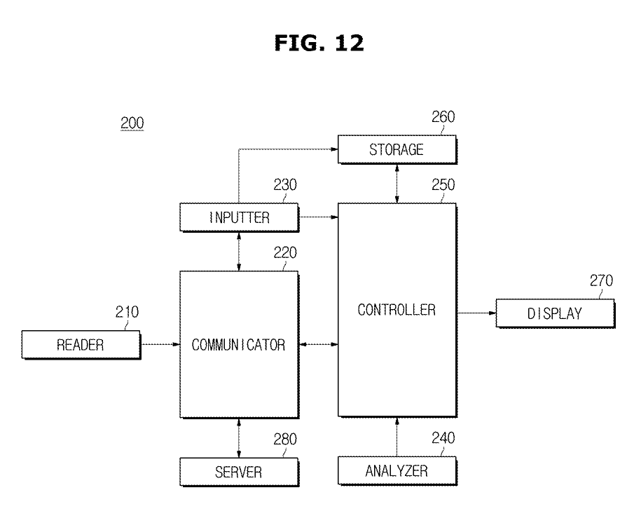

FIG. 12 is a block diagram illustrating elements of the pet blood test system 200 according to another exemplary embodiment.

Referring to FIG. 12, the pet blood test system 200 may include a reader 210 configured to read identification information of a pet, a communicator 220 configured to receive body information of the pet from a server 280 on the basis of the identification information of the pet, an inputter 230 configured to receive body information of the pet from a user when the body information of the pet is not present in the server 280, an analyzer 240 configured to analyze blood collected from the pet and acquire information on the blood, a controller 250 configured to provide the user with a result of a desired type of blood test on the basis of the body information of the pet received by the communicator 220 and blood information analyzed by the analyzer 240, a storage 260 configured to store a blood analysis result from the analyzer 240 and a blood test result, and a display 270 configured to display a result of a blood test in the controller 250 to the outside.

FIG. 12 illustrates an exemplary embodiment in which the server 280 is further added to the pet blood tester 100 illustrated in FIG. 2, which is the disclosure related to a system.

Because the elements in FIG. 12 are the same as those described with reference to FIG. 2 except the server 280, the pet blood test system 200 may include all features and advantageous effects of the pet blood tester 100 described with reference to FIG. 2. Consequently, because the pet blood tester 100 has been described above, the description thereof will be omitted, and features of the pet blood test system 200 focused on the server 280 and blood donation and blood transfusion service using the server 280 will be described.

The server 280 may store information of a pet blood tester that is different from the pet blood tester 100. Results of various tests performed by the pet blood tester 100 and a result of a blood test on another animal performed in another veterinary clinic may be shared through the server 280.

In this case, because pieces of information on other animals' blood type or disease recorded in another veterinary clinic can be shared, various services can be provided to the user.

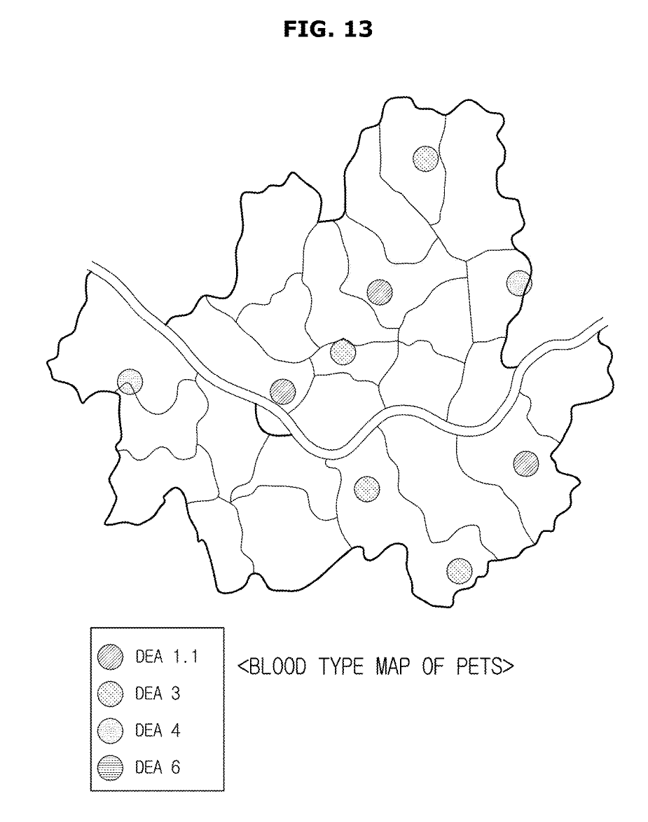

For example, a pet blood type map may be formed as illustrated in FIG. 13, blood type distribution for each veterinary clinic for each region may be stored and researched, and information on blood donation and blood transfusion of pets can be shared.

In the case of animals, particularly dogs, because blood type correlations that allow blood donation and blood transfusion are more complex than for humans, a blood type map has an advantageous effect of allowing blood donation to be more rapidly performed by immediately providing blood donation information to users treating animals that require an urgent blood donation.

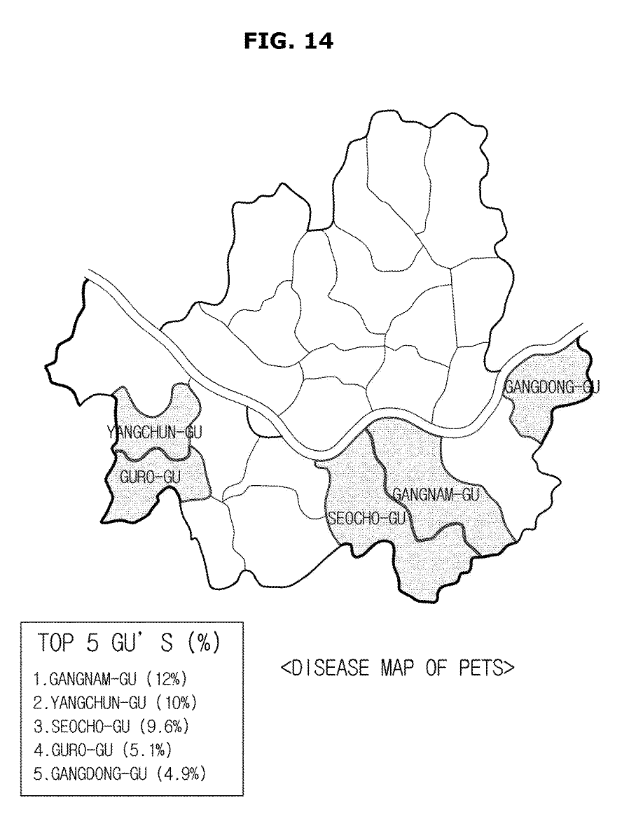

Also, as illustrated in FIG. 14, pet diseases in another region stored in the server 280 may be investigated and provided to the user as a pet disease map. Consequently, the user may determine which disease is prevalent in which region by the pet disease map so that preventive treatment can be more effectively performed on a pet, and the user can analyze the incidence of disease with respect to location.

Also, the pet blood test system 200 is not limited to providing services illustrated in FIGS. 13 and 14, but may also provide a service related to medical information of a pet to a user after pet information network is formed in connection with a clinic and the like using the server 280 and provide a pet owner with a service related to health management information of a pet.

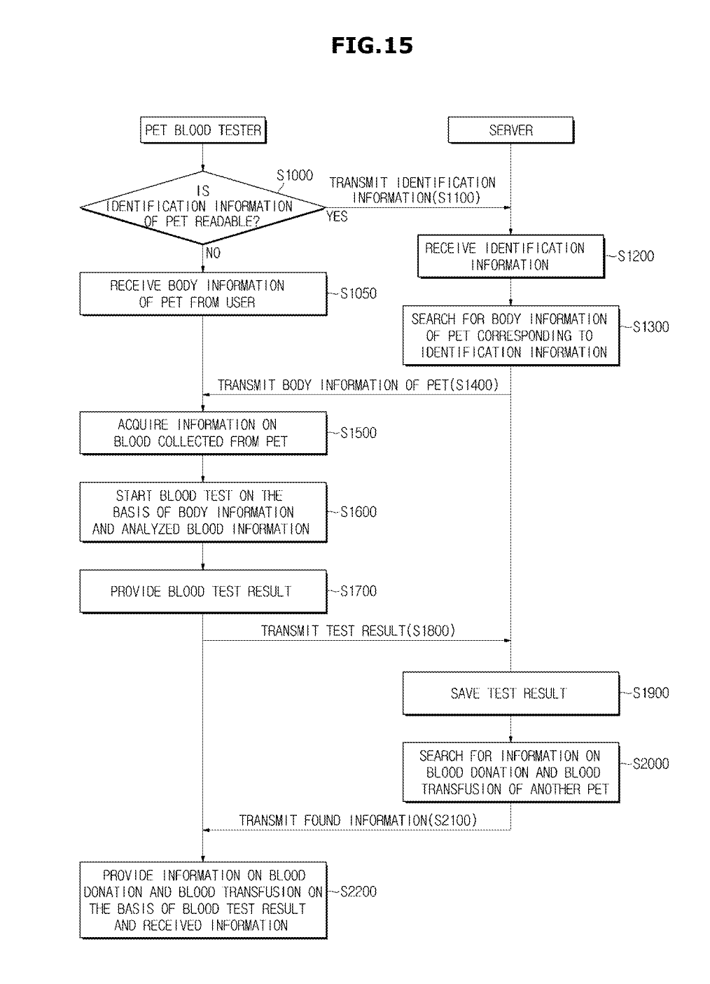

FIG. 15 is a flowchart illustrating an operation flow of the pet blood test system 200 illustrated in FIG. 12.

Referring to FIG. 15, the pet blood tester 100 may sense identification information of an animal using a tag of the animal (S1000).

When the identification information of the animal is not read, body information of the animal is directly received from a user (S1050), and when the identification information of the animal is read, the read identification information may be transmitted to a server (S1100).

Also, the server 280 may receive the identification information (S1200), and search for body information of the animal corresponding to the identification information (S1300), and then transmit the found body information of the animal to the pet blood tester 100 (S1400).

Because the pet blood test system 200 can receive body information of the animal from the server, an inconvenience of having to input the body information every time a blood test is performed can be solved.

When the body information of the animal is received, blood collected from the pet can be analyzed to acquire information on the blood (S1500).

Then, when information on the collected blood is acquired, various types of blood test results including blood type information on the basis of the received body information of the pet and the analyzed blood information can be provided to the user (S1600, S1700).

Then, the pet blood tester 100 may transmit blood type information and various types of test results to the server 280 (S1800), and the server 280 may receive and store the test results (S1900).

Then, when a command to search for blood type information or information on blood donation and blood transfusion of another pet is received from the user, the server 280 may search for blood type information or information on blood donation and blood transfusion of another animal (S2000) and then transmit the found information to the pet blood tester 100 (S2100).

Then, the pet blood tester 100 may provide the user with the information on blood donation and blood transfusion on the basis of the blood test result and the received information (2200).

Although FIG. 15 has been described on the basis of the information on blood donation and blood transfusion, the pet blood test system 200 is not limited thereto, and may also provide information on a pet's disease to the user as illustrated in FIG. 14 and provide medical information service of a pet through a network.

In the case of the related art, because a user has to directly input information on a pet, there is a problem in that an incorrect test result may be shown to the user due to a difference between pet information input by the user and information on a pet to be actually tested, and the problem causes misdiagnosis of a tested pet and has a negative influence on treating a pet.

Also, there is an inconvenience in that a user has to input pet information again when a veterinary clinic in which a pet is treated is changed, and because previous treatment results of a pet cannot be utilized, there are many problems.

However, because a pet blood tester according to the present disclosure can easily receive pet information and previous treatment results of a pet from an external device and perform a blood test on the pet on the basis thereof, there is an advantageous effect in that an accurate blood test result can be more quickly provided to a user.

Also, blood donation information and various pieces of treatment information required to a pet can be provided using a blood test result of the pet, and medical information service for the pet can be provided through a network.

Although exemplary embodiments have been described above with reference to the drawings, one of ordinary skill in the art should be able to modify and change the embodiments in various ways from the description above. For example, the techniques described above may be performed in a different order, and/or the above-described elements such as a system, a structure, a device, and a circuit may be coupled or combined in a different form from the above-described method, or an appropriate result may be achieved even when the elements are replaced or substituted by other elements or their equivalents. Therefore, other implementations, embodiments, claims, and their equivalents also belong to the scope of the claims below.

What is claimed is:

1. A pet blood tester comprising:
a reader configured to read identification information of a pet from a tag;
an analyzer configured to analyze blood of the pet to acquire information on the blood;
a storage;
a controller configured to receive stored body information of the pet corresponding to the identification information from at least one of an external device and the storage if the stored body information of the pet corresponding to the identification information is stored in at least one of the external device and the storage; and
an inputter configured to receive input body information of the pet from a user if the body information of the pet corresponding to the identification information is not stored in at least one of the external device and the storage,
wherein the controller is further configured to provide a blood test result and diagnosis information for the pet, based on the information on the blood, and the stored body information of the pet or the input body information on the blood.

2. The pet blood tester of claim 1, wherein the controller is further configured to receive the stored body information of the pet that is most recently stored or is selected according to a standard preset by the user, when the same stored body information of the pet is stored in the external device and the storage.

3. The pet blood tester of claim 2, wherein the controller is further configured to control the external device to store in the external device the blood test result of the pet, and the input body information of the pet or the stored body information of the pet stored in the storage.

4. The pet blood tester of claim 1, wherein the body information includes at least one of a name, a species, a sex, a blood type, an age, a height, and a weight of the pet.

5. The pet blood tester of claim 1, wherein the blood test result includes at least one of a blood type test result, a clinical chemistry test result, and an electrolyte test result.

6. The pet blood tester of claim 1, wherein the identification information includes at least one of a barcode, a quick response code, text data, radio frequency identification information, and a recognition pattern.

7. The pet blood tester of claim 1, wherein the reader includes at least one of a barcode reader, a radio frequency identification reader, a near-field communication sensor, and an image sensor.

8. The pet blood tester of claim 1, wherein the storage stores the input body information received from the user or information received from the external device.

9. The pet blood tester of claim 1, further comprising a display configured to display a state of progress of analysis or to display at least one of the blood test result and the diagnosis information.

10. The pet blood tester of claim 1, wherein the controller is further configured to receive, from the external device, information on another pet present in a veterinary clinic within a predetermined area.

11. The pet blood tester of claim 10, wherein the controller is further configured to determine whether a blood donation from the pet to the other pet or a blood transfusion to the pet from the other pet is possible based on the information on the other pet received from the external device.

12. The pet blood tester of claim 10, wherein the controller is further configured to provide at least one of a blood type map, disease information, and clinic information of at least one pet on based on the information on the other pet received from the external device.

13. A control method of a pet blood tester, the control method comprising:
reading identification information of a pet from a tag;
analyzing blood to acquire information on blood of the pet;
receiving stored body information of the pet corresponding to the identification information if the stored body information of the pet corresponding to the identification information is stored in at least one of an external device and a storage of the pet blood tester;
receiving input body information of the pet from a user if the stored body information is not stored in at least one of the external device and the storage of the pet blood test; and
providing the user with a blood test result and diagnosis information for the pet based on the information on the blood, and the stored body information of the pet or the input body information of the pet from the user.

14. The control method of claim 13, further comprising storing the input body information of the pet and the blood test result of the pet.

15. The control method of claim 13, wherein the body information includes at least one of a name, a species, a sex, a blood type, an age, a height, and a weight of the pet.

16. The control method of claim 15, further comprising determining whether a blood donation from the pet to another pet or a blood transfusion to the pet from the other pet is possible based on blood type information of the pet and blood type information of the other pet that is stored in the external device.

17. The control method of claim 13, wherein the reading of the identification information includes reading the identification information with at least one of a barcode reader, a radio frequency identification reader, a near-field communication sensor, and an image sensor.

18. The control method of claim 13, further comprising displaying a state of progress of the analyzing or displaying at least one of the blood test result and the diagnosis information.

19. The control method of claim 13, wherein the receiving the stored body information of the pet includes receiving, from the external device, information on a species, a sex, a blood type, a blood donation, and a blood transfusion of another pet present in a veterinary clinic within a predetermined area.

20. The control method of claim 19, further comprising providing the user with information indicating whether a blood donation from the pet to the other pet or a blood transfusion to the pet from the other pet is possible based on the information on the other pet received from the external device.
